(19) United States
(12) Patent Application Publication
(10) Pub. No.: US 2007/0282336 A1
(43) Pub. Date: Dec. 6, 2007

(54) Bipolar high-frequency treatment tool for endoscope

(75) Inventor: Tomohiro Kawano, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/442,418

(22) Filed: May 30, 2006

(51) Int. Cl. A61B 18/18

(52) U.S. Cl. 606/51

(57) ABSTRACT

A high-frequency treatment tool for an endoscope includes a connector configured to be connected to an inserting portion of an endoscope and further configured to be inserted into a body cavity through an endoscope, and first and second electrodes mounted to a distal end of the connector and movable between an open position and a closed position.. The first electrode is connected, at a proximal end, with a conductive wire configured to be provided with high frequency voltage of a first polarity, the second electrode is connected, at a proximal end, with a conductive wire configured to be provided with high frequency voltage of a second polarity, the first and second electrodes each have a tip portion extending at an angle from the respective first and second electrodes, and the opposed surfaces of the first and second electrodes are positioned closer to each other in the closed position than in the open position.

### BACKGROUND OF THE INVENTION

1. Field of the Invention

The present invention is related to bipolar high-frequency treatment tool, and more particularly relates to a multipurpose treatment tool for an endoscope

2. Background and Material Information

Bipolar electrosurgical tools have become increasingly popular because of their safety over monopolar electrosurgical tools, resulting in a corresponding increase in the variety of surgical procedures performed by such bipolar electrosurgical tools. Such purpose-built tools may be used for marking, incision, exfoliation or hemostasis. However, since a different tool must be used for each procedure, the surgeon is required to maintain a variety of tools in inventory (resulting in increased costs), and the duration of the surgical treatment is increased due to the time it takes to exchange tools during surgery (thereby increasing the risk to the patient). Therefore, a need has arisen for a multipurpose bipolar high-frequency treatment tool that can perform a variety of different surgical procedures

### SUMMARY OF THE INVENTION

A non-limiting feature of the invention provides a multipurpose bipolar high-frequency treatment tool that can perform a variety of different surgical procedures, including but not limited to marking, incision, exfoliation or hemostasis, thereby reducing the duration of surgery (thereby reducing the risk to the patient) and reducing the number of surgical tools needed for surgery (thereby reducing costs).

A non-limiting embodiment of the present invention provides a high-frequency treatment tool for an endoscope, having a connector configured to be connected to an inserting portion of an endoscope and further configured to be inserted into a body cavity through an endoscope, and first and second electrodes mounted to a distal end of the connector and movable between an open position and a closed position. The first electrode may be connected, at a proximal end, with a conductive wire configured to be provided with high frequency voltage of a first polarity, the second electrode is connected, at a proximal end, with a conductive wire configured to be provided with high frequency voltage of a second polarity, the first and second electrodes may each have a tip portion extending at an angle from the respective first and second electrodes, and the opposed surfaces of the first and second electrodes may be positioned closer to each other in the closed position than in the open position.

In another feature of the invention, the angle may be approximately 90 degrees and/or may form approximately an L-shape with the respective first and second electrodes. A further feature of the invention may include teeth on at least one of the opposed surfaces of the first and second electrodes.

The first and second electrodes may also be pivotable between the open position and the closed position. There may also be a gap between the opposed surfaces of the first and second electrodes in the closed position. In another feature of the invention, a blade may be formed on a trailing edge of at least one tip of the first and second electrode and/or a blade may be formed on a leading edge of at least one tip of the first and second electrodes. Further, the tip portions may be rounded.

A further feature of the invention provides a high-frequency treatment tool for an endoscope, having a tool support configured to be connected to an inserting portion of an endoscope and further configured to be inserted into a body cavity through an endoscope, and an operable electrode assembly. The assembly may include a body having a proximal end and a distal end, the proximal end mounted to a distal end of the tool support, a tip portion extending at an angle from the proximal end of the body, a first electrode connected, at a proximal end, with a conductive wire configured to be provided with high frequency voltage of a first polarity, and a second electrode connected, at a proximal end, with a conductive wire configured to be provided with high frequency voltage of a second polarity, the first and second electrodes movable between an open position and a closed position. The opposed surfaces of the first and second electrodes are positioned closer to each other in the closed position than in the open position.

Other exemplary embodiments and advantages of the present invention may be ascertained by reviewing the present disclosure and the accompanying drawings, and the above description should not be considered to limit the scope of the present invention.

### BRIEF DESCRIPTION OF THE DRAWINGS

The present invention is further described in the detailed description which follows, in reference to the noted plurality of drawings, by way of non-limiting examples of preferred embodiments of the present invention, in which like characters represent like elements throughout the several views of the drawings, and wherein:

FIG. 1 shows a perspective view of the high-frequency treatment tool in a closed position, in accordance with a non-limiting embodiment of the present invention;

FIG. 2 shows a side section al view of the tool in an open position, taken along the line A-A of FIG. 3;

FIG. 3 shows a top plan view of the tool;

FIG. 4 shows a perspective view of the tool in a marking procedure,

FIG. 5 shows a perspective view of the tool in a marking procedure;

FIG. 6 shows a perspective view of the tool in an incisional procedure;

FIG. 7 shows a side schematic view of the tool in a closed position in a hemostasis procedure;

FIG. 8 shows a side schematic view of the tool in an open position in a hemostasis procedure;

FIG. 9 shows a side schematic view of the tool in an open position in a hemostasis procedure;

FIG. 10 shows a side schematic view of the tool in a closed position in a hemostasis procedure,

FIG. 11 shows a perspective schematic view of the tool in an open position in an incisional procedure;

FIG. 12 shows a perspective schematic view of the tool in a closed position in an incisional procedure;

FIG. 13 shows a perspective schematic view of the tool in a closed position in an incisional procedure; and

FIG. 14 shows a perspective schematic view of the tool in a closed position in an incisional procedure.

### DETAILED DESCRIPTION OF THE PREFERRED EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

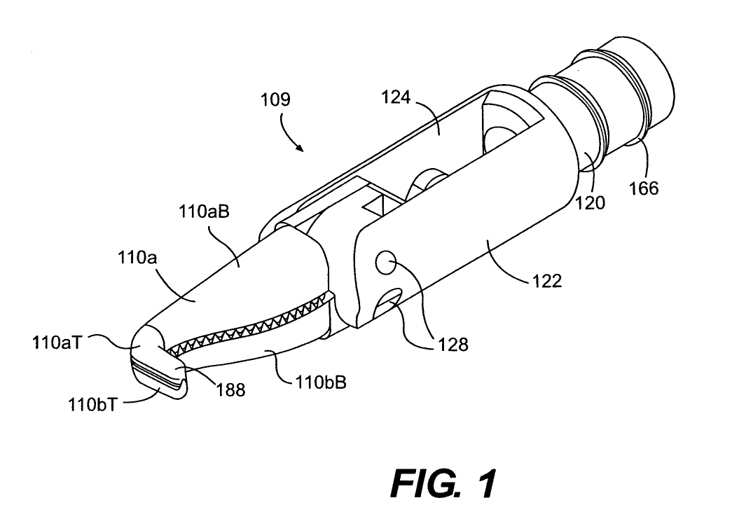

Referring to the drawings, wherein like characters represent like elements, FIG. 1 shows a perspective view of a bipolar high-frequency treatment tool 109 for an endoscope according to a non-limiting embodiment of the present invention. The tool may be used in conjunction with a bipolar high-frequency endoscopic surgical system described, for example, in U.S. Pat. No. 6,969,389 and U.S. Patent Publication No. 2003/0191465, both disclosures being expressly incorporated herein by reference.

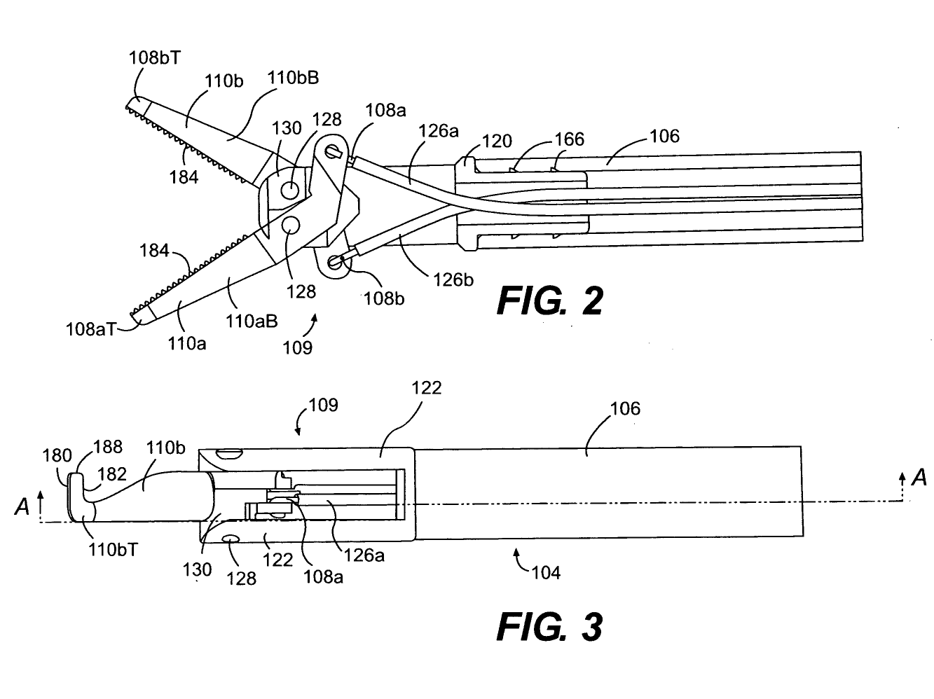

The tool 109 has a connector 120 (also referred to as a “tool support”) configured to be connected to an inserting portion 104 (shown in FIGS. 2-3) configured to be inserted into a body cavity through the endoscope. The inserting portion 104 is provided in a form and size that allows it to be introduced into a body cavity through a treatment tool inserting channel of an endoscope (not shown). The inserting portion 104 includes an elongated and flexible sheath 106, a pair of conductive wires 108a, 108b slidably inserted through the sheath 106, and a pair of electrodes 110a, 110b provided at the distal end of the tool support 120 and connected to the conductive wires 108a, 108b. In this fashion, each electrode is of a different polarity. For example, electrode 110a may have a positive polarity, and the electrode 110b may have a negative polarity, or vice versa. The electrodes are movable between a closed position (when the conductive wires 108a, 108b are moved proximally, as shown in FIG. 1) and an open position (when the conductive wires 108a, 108b are moved distally, as shown in FIG. 2).

The sheath 106 may be made of insulating material such as poly-tetra-fluoro-ethylene (PTFE), although those skilled in the art will readily understand that the sheath may be made of any other suitable material. While FIG. 2 shows the tool support 120 press-fit into the sheath 106 via rings 166, those skilled in the art will readily understand that the tool support may be secured in the sheath by a variety of other suitable ways, including but not limited to threading, snap-fitting, and the like.

The supporting member 120 may be made of hard insulating material such as rigid plastic, although those skilled in the art will readily understand that the supporting member may be made of any other suitable material. The supporting member 120 has two arms 122 extending forwards and generally parallel to each other to form a slit 124. Two pins 128 are supported between the arms 122 in the vicinity of the distal end thereof. The pins 128 are arranged generally parallel to and spaced apart from each other, and perpendicular to the side walls of the slit 124.

The pair of electrodes 110a, 110b are partially inserted into the slit 124 of the supporting member 120 and are pivotably mounted to the pair of pins 128. Thus, the pair of electrodes 110 can move between the closed position shown in FIG. 1, and the open position shown in FIG. 2, in which the electrodes 110a, 110b are located further apart from each other than when they are in the closed position. In an embodiment, the electrodes 110a, 100b (which have polarities different from each other) do not come into contact with each other in the closed position, in order to prevent over-pinching of the tissue which may otherwise result in injury to the patient. The resulting space between the electrodes 110a, 110b in the closed position may range from, for example, approximately 0.05 mm to approximately 0.5 mm. In another embodiment, however, the electrodes 110a, 100b of differing polarities may come into contact with each other in the closed position, in situations where such a configuration is desirable

The rear ends or proximal ends of the electrodes 110a, 110b are respectively connected with the conductive wires 108a, 108b. Each of the conductive wires 108a, 108b is covered with an insulating tube 126a, 126b except the end portion thereof at which the conductive wire 108a, 108b is connected to the corresponding electrode 110a, 110b.

An insulating block 130 is located in the slit 124 of the supporting member 120 to prevent the electrodes 110 from coming into contact to each other within the slit 124. The insulating block 130 is located between the electrodes 110 and supported by the pins 128. The insulating block 130 may be made of resin such as poly-tetra-fluoro-ethylene, for example, although those skilled in the art will readily understand that the insulating block may be made of any other suitable material.

The electrodes 110a, 110b are elongated members and may be made of metal such as stainless steel, although those skilled in the art will readily understand that the electrodes may be made of any other suitable material. The electrodes 110a, 110b each include a body 110aB, 110bB and a tip portion 110aT, 110bT extending at an angle from the distal end of the body 110aB, 110bB. In a non-limiting embodiment, the tip portion 110aT, 110bT extends at generally a 90 degree angle to the distal end of the body 110aB, 110bB, and to form a general L-shape, although those skilled in the art will readily understand that the tip may form other angles with the body, depending on the needs of the surgeon.

Also, in order to improve cutting and/or current density, the tip 110aT, 110bT may include a blade 180 at the leading edge thereof and/or a blade 182 at the trailing edge thereof, depending on the needs of the surgeon. Additionally, in order to prevent slippage of the tool 109, the electrode 110a, 110b may include teeth 184 on one or both of the opposed inner surfaces thereof and which may or may not interlock, depending on the needs of the surgeon. Further, to prevent tissue damage and/or to improve tissue marking, the end 186 of the tip 110aT, 110bT may be rounded or blunt.

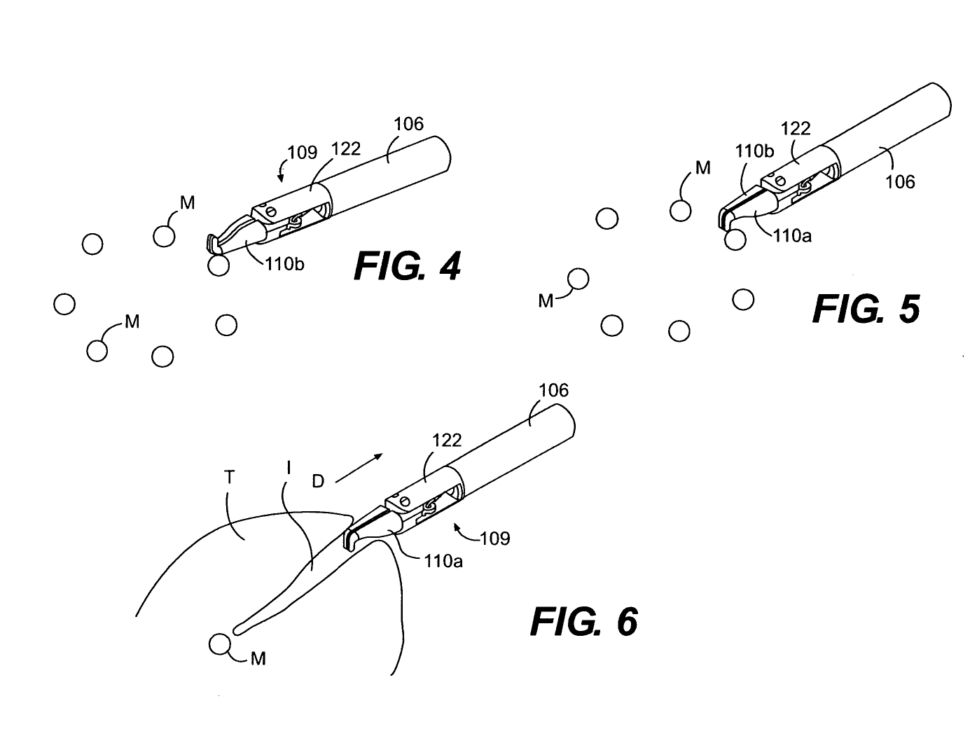

FIGS. 4-5 show the tool 109 used in a tissue marking operation, where a surgeon may mark tissue T for treatment. The tool 109 may be rotated from the position shown in FIG. 4, to the position shown in FIG. 5, where the tip portion 110aT, 110bT is pointed downward toward the tissue T. The surgeon may then apply electric current to the tool 109 while applying the tip portion 110aT, 110bT to the tissue T, which is seared due to the heat generated by the current, thereby creating a mark M on the tissue.

Once the tissue T is marked, a surgical procedure (e.g., mucosal or submucosal incision or dissection) may be performed, as shown in FIG. 6. The surgeon may apply the tip portion 110aT, 110bT to the tissue T while applying electric current to the tool 109. The tool 109 may then be moved proximally along direction D, thereby creating an incision I in the tissue T.

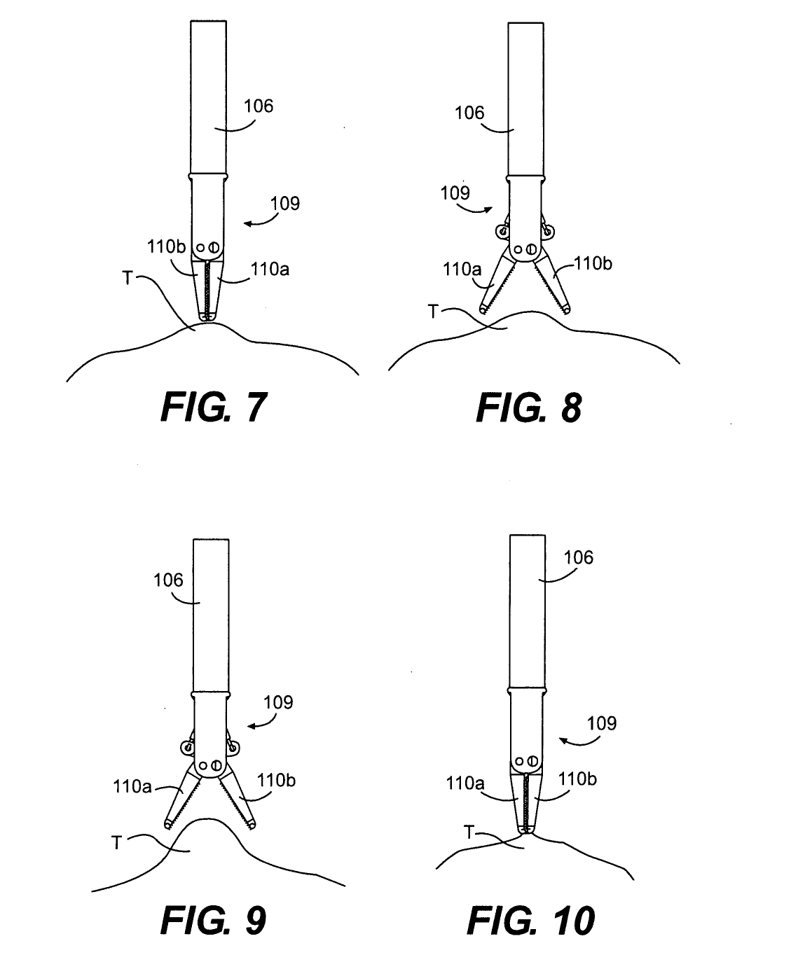

As shown in FIG. 7, to stop the bleeding of tissue T in a relatively concentrated area, the surgeon may apply the tool 109 with the electrodes 110a, 100b in a closed position, while applying electric current to the tool. As shown in FIG. 8, in order to stop the bleeding of tissue T over a relatively wide area, the surgeon may apply the tool 109 with the electrodes 110a, 100b in an open position. As shown in FIGS. 9-10, the surgeon may then pinch the electrodes 110a, 110b over the bleeding tissue T, while applying electric current to the tool, thereby stopping bleeding of the tissue.

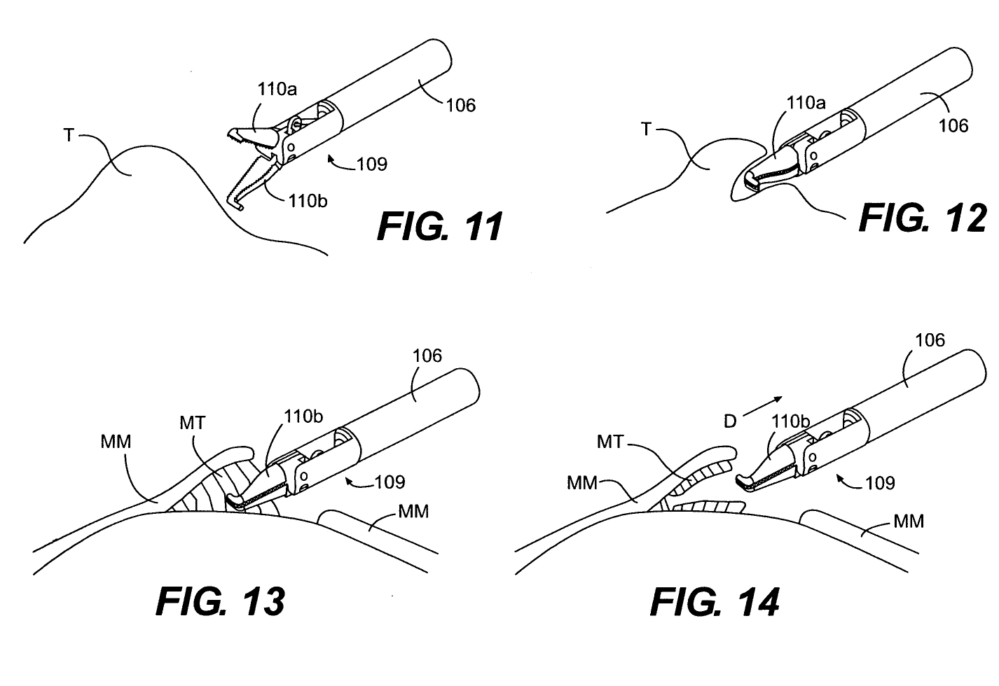

As shown in FIGS. 11-12, the tool 109 may be used for pinching and incision. For example, the surgeon may pinch the affected tissue T between the electrodes 110a, 110b while applying electric current to the tool 109, thereby incising the tissue T.

Further, as shown in FIGS. 13-14, the tool 109 may also be used for incision of muscle layer tissue MT of the muscle layer ML, located under the mucous membranes MM. For example, as shown in FIG. 13, the surgeon may select a desired amount of muscle layer tissue MT with the tip portion 110aT, 110bT of the respective electrodes 110a, 110b (in a closed position). Once the proper amount of muscle layer tissue MT has been selected, as shown in FIG. 14, electric current is applied to the tool 109 while it is pulled in direction D, such that the trailing end of the tip portion 110aT, 110bT engages and incises the muscle layer tissue MT.

By the above-described configuration, the high-frequency treatment tool 109 can perform a variety of different surgical procedures, including but not limited to marking, incision, exfoliation or hemostasis, thereby reducing the duration of surgery (thereby reducing the risk to the patient) and reducing the number of surgical tools needed for surgery (thereby reducing costs).

It is noted that those skilled in the art will readily understand that to assist the surgeon in performing any of marking, incision, exfoliation and hemostasis with the tool 109 of the present invention, different qualities of high-frequency voltage may be used (e.g., different duration, voltage and the like)

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A high-frequency treatment tool for an endoscope, comprising:
a connector configured to be connected to an inserting portion of an endoscope and further configured to be inserted into a body cavity through an endoscope; and
first and second electrodes mounted to a distal end of said connector and movable between an open position and a closed position,
wherein:
said first electrode is connected, at a proximal end, with a conductive wire configured to be provided with high frequency voltage of a first polarity,
said second electrode is connected, at a proximal end, with a conductive wire configured to be provided with high frequency voltage of a second polarity;
said first and second electrodes each have a tip portion extending at an angle from said respective first and second electrodes; and
said opposed surfaces of said first and second electrodes are positioned closer to each other in the closed position than in the open position.

2. The tool according to claim 1, wherein the angle is approximately 90 degrees.

3. The tool according to claim 1, further comprising teeth on said opposed surfaces of said first and second electrodes.

4. The tool according to claim 1, wherein said first and second electrodes are pivotable between the open position and the closed position.

5. The tool according to claim 1, further comprising a gap between the opposed surfaces of said first and second electrodes in the closed position.

6. The tool according to claim 1, further comprising a blade formed on a trailing edge of at least one said tip of said first and second electrodes.

7. The tool according to claim 6, further comprising a blade formed on a leading edge of at least one said tip of said first and second electrodes.

8. The tool according to claim 1, further comprising a blade formed on a leading edge of at least one said tip of said first and second electrodes.

9. The tool according to claim 1, wherein said tip portions form approximately an L-shape with said respective first and second electrodes

10. The tool according to claim 1, wherein said tip portions are rounded.

11. A high-frequency treatment tool for an endoscope, comprising.
a tool support configured to be connected to an inserting portion of an endoscope and further configured to be inserted into a body cavity through an endoscope; and
an operable electrode assembly comprising:
a body having a proximal end and a distal end, said proximal end mounted to a distal end of said tool support;
a tip portion extending at an angle from said proximal end of said body;
a first electrode connected, at a proximal end, with a conductive wire configured to be provided with high frequency voltage of a first polarity; and
a second electrode connected, at a proximal end, with a conductive wire configured to be provided with high frequency voltage of a second polarity, said first and second electrodes movable between an open position and a closed position,
wherein opposed surfaces of said first and second electrodes are positioned closer to each other in the closed position than in the open position.

12. The tool according to claim 11, wherein the angle is approximately 90 degrees.

13. The tool according to claim 11, further comprising teeth on said opposed surfaces of said first and second electrodes.

14. The tool according to claim 11, wherein said first and second electrodes are pivotable between the open position and the closed position.

15. The tool according to claim 11, further comprising a gap between the opposed surfaces of said first and second electrodes in the closed position.

16. The tool according to claim 11, further comprising a blade formed on a trailing edge of at said tip portion.

17. The tool according to claim 16, further comprising a blade formed on a leading edge of said tip portion.

18. The tool according to claim 11, further comprising a blade formed on a leading edge of said tip portion.

19. The tool according to claim 11, wherein said tip portion forms approximately an L-shape with said body.

20. The tool according to claim 11, wherein said tip portion is rounded.
